(19) United States
(12) Patent Application Publication
(10) Pub. No.: US 2025/0082826 A1
(43) Pub. Date: Mar. 13, 2025

(54) Compressed Collagen Composite Construct for Cell or Therapeutic Delivery

(71) Applicant: The Board of Trustees of the Leland Stanford Junior University, Stanford, CA (US)

(72) Inventors: Yunzhi Yang, Stanford, CA (US); Seyedsina Moeinzadeh, Mountain View, CA (US); Geoffrey Abrams, Menlo Park, CA (US)

(21) Appl. No.: 18/727,159

(22) PCT Filed: Jan. 27, 2023
(86) PCT No.: PCT/US2023/011700

§ 371 Date: Jul. 8, 2024

Related U.S. Application Data
(60) Provisional application No. 63/304,200, filed on Jan. 28, 2022.

(51) Int. Cl. A61L 27/48; A61L 27/52; A61L 27/54; A61L 27/56

(52) U.S. Cl. CPC A61L 27/48; A61L 27/52; A61L 27/54; A61L 27/56; A61L 2400/18; A61L 2430/24

(57) ABSTRACT

Technology is provided for the production of collagen-based composite grafts with robust mechanical properties exceeding that of typical collagen hydrogels or foams. Collagen is a biodegradable and biocompatible hydrogel, but its clinical application is limited by poor mechanical properties that hinders its manipulation during surgeries and its post-surgical stability. The new collagen composite construct has robust mechanical properties for easy surgical handling such as rolling, suturing and passing a small surgical port during arthroscopic procedure, while maintaining unique biological characteristics of collagen matrix. Cells from various sources, bone marrow aspirate concentrates, growth factors, nanoparticles/microparticles (NPs/MPs), and supportive polymer meshes can be incorporated into the membranes for drug delivery and tissue regenerative therapies.

### FIELD OF THE INVENTION

This invention relates to therapeutic delivery devices, methods of making therapeutic delivery devices and methods for the delivery of therapeutics.

### BACKGROUND OF THE INVENTION

Rotator cuff (RC) tears are one of the most common orthopedic injuries, resulting in shoulder pain and dysfunction. The unmet medical needs and clinical importance of this project are highlighted by the large number of rotator cuff surgeries (more than 200,000) and high cost (approximately $3.44 billion USD) per year in the United States alone.

Current surgical management of symptomatic RC tears is centered around re-attaching the torn RC tendon back to bone utilizing suture anchors through arthroscopic technique. When the tendon heals properly, the native RC bone-tendon interface (enthesis) is re-created, leading to a smooth transition from tendon, the un-mineralized and then mineralized fibrocartilage, and then bone over a distance of less than 1 mm.

Unfortunately, RC tendon re-tear rates following surgery can be up to 94%, due to an inferior biological healing environment leading to mechanically inferior fibrovascular scar tissue which is predisposed to tearing. These re-tears are a significant impediment to recovery of maximal shoulder function following surgery and remain an unsolved clinical problem.

Ultimately, the presence of mesenchymal stem cells (MSC), and the environment to which they are exposed, are responsible for tendon healing and enthesis regeneration after surgical repair.

Although commercially available rotator repair grafts have been introduced to augment the repair, they have been associated with poor clinical success. These grafts lack appropriate biological cues such as signaling molecules or stem cells at the bone-tendon interface. The present invention is intended to advance the art and in particular RC tears as one of the most common orthopedic injuries.

### SUMMARY OF THE INVENTION

This invention provides technology for the production of collagen-based composite grafts with robust mechanical properties exceeding that of typical collagen hydrogels or foams. Collagen is a biodegradable and biocompatible hydrogel, but its clinical application is limited by poor mechanical properties that hinders its manipulation during surgeries and its post-surgical stability. The new collagen composite construct according to this invention has robust mechanical properties for easy surgical handling such as rolling, suturing and passing a small surgical port during arthroscopic procedure, while maintaining unique biological characteristics of collagen matrix. Cells from various sources, bone marrow aspirate concentrates, growth factors, nanoparticles/microparticles (NPs/MPs), and supportive polymer meshes can be incorporated into the membranes for drug delivery and tissue regenerative therapies.

In one embodiment, the invention is characterized as a method of making a therapeutic collagen-based delivery platform. The method distinguishes the following steps:
having a mesh of struts;
treating a surface of the mesh to form a hydrophilic surface followed by coating with interactive molecules (e.g. reactive groups) for polymers to physically attach to the surface to enhance interaction with collagen, where the treated mesh has a thickness of 0.2 to 3 mm. Examples of interactive molecules are gelatin, gelatin methacrylate, collagen, collagen binding proteins or peptides. To clarify this is a two-step treatment. First one creates a hydrophilic surface of polyester by using acid or base to cleavage the amide bonds to provide free carboxylic group (—COOH) that helps physical interaction with gelatin or its-like, and then followed by coating with a gelatin and its-like to facilitate the binding of the gelatin and its-like to the following collagen matrix.
embedding the treated mesh in a collagen hydrogel forming a composite construct, where the collagen hydrogel has a thickness of 4 to 20 mm larger, and where the collagen hydrogel is loaded with therapeutics; and
placing the composite construct between an absorbable medium such as filter papers and its-like. In the method, the filer papers help remove the water during compression.
compressing the collagen hydrogel such that the composite construct has a thickness of 0.2 to 3 mm, plus not more than 300, 400, 600 or 900 micrometers. The objective of the compression and absorption is to gain mechanical strength while retaining biological properties of the biological-laden composite which is accomplished by quickly removing water and reducing the thickness of the collagen hydrogel.

In an additional embodiment, the method is further characterized by freeze-drying the composite construct. Freeze Drying is a process in which a completely frozen sample is placed under a vacuum to remove water or other solvents from the sample, allowing the ice to change directly from a solid to a vapor without passing through a liquid phase.

In yet another embodiment, the method is further characterized by coating the composite construct.

In still another embodiment, the method is further characterized by embedding the composite construct in an integrated sponge, in which the composite as fabricated described herein is embedded in a collagen-based hydrogel followed by zero to 10 min compression and then followed by a freeze-drying to form a singular dense-porous integrated collagen structure. The fabrication steps such as collagen embedding, compression and freeze-drying in their entirety or in part could be repeated as needed to tune their properties. The absence of compression or shorter compression will retain the pores or porous structure of the sponge layer during freeze-drying. The highly porous structure of the sponge layer will help contain added stem cells or bone marrow aspirate concentrate during implantation and cell migration and tissue ingrowth after implantation.

The core, i.e. the therapeutic collagen-based delivery platform is always compressed. 4C is referred to as that core. 4C can be used to deliver cells, growth factors and drugs by itself. 5C is the core plus additional collagen layer(s) regardless of compression. When 5C is used for cells and growth factor delivery, in general, but not limiting to, 4C or the core is loaded with growth factor and controls its release, and the additional collagen layer(s) of 5C is loaded with cells and retains cells during implantation. Combining growth factor effect by 4C as the core and cell effect by additional collagen layer(s) of 5C leads to better e.g. rotator cuff repair (or the like) and regeneration. Generally, therapeutic collagen-based delivery platforms according to the invention can be used to deliver cell and biologics for regeneration and/or repair, including bone and soft tissue such as heart, muscle, cartilage and/or skin. In addition, there are still other possibilities such as e.g., but not limiting to, additional collagen layer(s) in 5C which could have cells and growth factors for regeneration and/or repair.

In other embodiment, several steps of the method can be repeated or varied. For example:
One could compress and freeze-dry the composite to form the 4C construct, and then re-immerse the 4C into the collagen hydrogel again.
One could compress and freeze-dry the composite to form the 4C construct, and then re-immerse the 4C into the collagen hydrogel and repeat the compression and freeze-drying again.
One could not compress and just freeze dry the 4C with the collagen hydrogel to form 5C of a single layered collagen sponge.
One could re-immerse the 4C into the collagen hydrogel and repeat compression and freeze-drying, to then get 5C of variety, which means a 5C with multi-layered collagen, in which only the outermost layer of collagen is uncompressed and forms the sponge, and the rest of the middle layers are compressed and form the dense layers.

Examples of therapeutics are cells, drugs, proteins, peptides, small molecules, RNA, or particles loaded with drugs, proteins, small molecules, peptides, or RNA.

The treating steps in the method are crucial for the mechanical properties and structural integrity during surgical handling and implantation. Without such steps, the composite cannot be made because the collagen component is easily detached from the polymeric mesh. Without such the steps, even if the composite could be made, the structural integrity of such the composite implant is brittle, easily broken and not suitable for surgical handling and implantation.

The therapeutic collagen-based delivery platform produced by the method can used for regeneration or repair of an orthopedic injury or a soft tissue injury, or, but not limiting to, for regeneration or repair of a rotator cuff repair. However, therapeutic collagen-based delivery platform produced by the method is not limited to these applications as it can have other applications where therapeutic delivery of cell and/or biologics plays a role for tissue regeneration or repair.

In still another embodiment, a therapeutic collagen-based delivery platform for regeneration and/or repair of an orthopedic injury or a soft tissue injury is provided. The therapeutic collagen-based delivery platform has a mesh of polymer struts having a hydrophilic surface and interactive surface molecules, where the mesh of polymer struts is embedded in a compressed collagen hydrogel, where the collagen in the collagen hydrogel is physically attached to polymers at the mesh surface through interactive molecules, wherein the thickness of the mesh of polymer struts is only increased by up to 300, 400, 600 or 900 micrometers as a result of the embedded and compressed collagen hydrogel.

### BRIEF DESCRIPTION OF THE DRAWINGS

FIGS. 1A-F show according to exemplary embodiments of the invention a schematic representation of the compressed collagen synthesis process (FIG. 1A), an image of the compressed collagen microsheet (FIG. 1B), the effect of compression time on the thickness of the compressed collagen microsheet (FIG. 1C), the effect of compression time on the viability of MSCs encapsulated on compressed collagen microsheet (FIG. 1D), the proliferation of MSCs and keratinocytes encapsulated in compressed collagen microsheet (FIGS. 1E-F).

FIG. 2 shows according to an exemplary embodiment of the invention H&E (Hematoxylin and eosin stain, top row) and human nuclear antigen staining (bottom row) of human MSCs delivered in Matrigel, injectable collagen-based gel, and compressed collagen microsheet to NSG mice after 8 weeks of subcutaneous implantation.

FIG. 3 shows according to an exemplary embodiment of the invention (top) schematic representation of 4C synthesis method. (bottom) 4C made using PCL and PCL/GelMA mesh.

FIGS. 4A-F show according to exemplary embodiments of the invention in FIG. 4A live (green as shown in same figure in the priority document) and dead (red as shown in same figure in the priority document) human MSC cells encapsulated in 4C after 7 days of incubation (FIG. 4B) viability of human MSCs in 4C over 7 days (FIG. 4C) an image of a cell-laden 4C right after making the construct (FIGS. 4D-E) images of an hMSC cell-laden 4C after 21 days of incubation (FIG. 4F) proliferation of hMSCs in 4C over 21 days.

FIGS. 5A-D show according to exemplary embodiments of the invention in FIG. 5A effect of compression on the retention of FITC (Fluorescein isothiocyanate)-labeled BSA (bovine serum albumin) protein in 4C constructs (FIG. 5B) release kinetics of FITC-labeled BSA protein from 4C constructs in the absence or presence of collagenase enzyme, or from modified 4C (m4C) in the presence of enzyme (FIG. 5C) effect of compression time on the BMP2 protein retention in 4C constructs (FIG. 5D) release kinetics of BMP2 from 4C constructs in the absence or presence of collagenase enzyme, or from modified 4C (m4C) in the presence of enzyme.

FIGS. 6A-E show according to exemplary embodiments of the invention in FIG. 6A fresh 5C (FIG. 6B, 6C) freeze-dried 5C (FIG. 6D) freeze dried 5C loaded with hMSC cells (FIG. 6E) effect of hMSC cell density on the retention of cells on 5C constructs. Ctrl group in (FIG. 6E) shows the measured number of cells suspended in the cell culture medium.

FIGS. 7A-B show according to exemplary embodiments of the invention in FIG. 7A grafting a 4C polymer mesh on a cadaveric rabbit rotator cuff defect model (FIG. 7B) arthroscopically insertion of a 5C in a human cadaveric shoulder.

FIGS. 8A-d show according to exemplary embodiments of the invention in FIG. 8A a bovine-based 4C, (FIG. 8B) live (green as shown in same figure in the priority document) and dead (red as shown in same figure in the priority document) hMSC cells encapsulated in a bovine-based 4C after 1 day of incubation (FIG. 8C) viability of hMSCs in rat-based and bovine-based 4C after 1 day on incubation (FIG. 8D) proliferation of hMSCs in rat-based and bovine-based 4C after 21 days.

FIG. 9 shows according to an exemplary embodiment of the invention a fresh-frozen and freeze-dried 4C or 5C composites in the presence and absence of additional single or three-layered hydrophobic PCL coating with different parameters.

FIG. 10 shows according to an exemplary embodiment of the invention feasibility of and the retention of structural integrity of 4c or 5c composites during surgical manipulation.

FIG. 11 shows according to an exemplary embodiment of the invention a fresh, or freeze-dried 5c composite, the suitability of surgical manipulation of the 5c, and the arthroscopic implantation of composite into a cadaver shoulder.

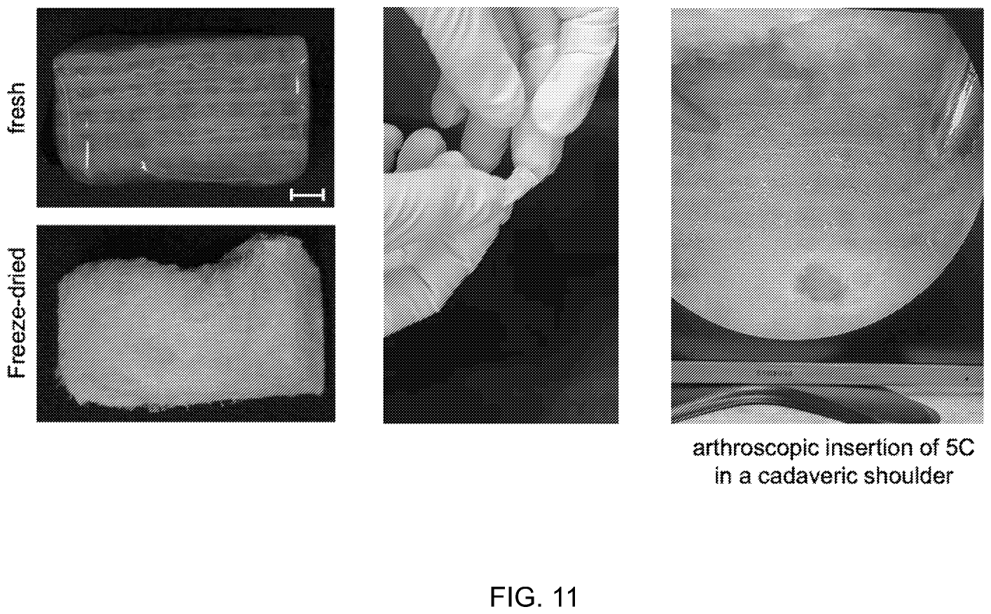

FIG. 12 shows according to an exemplary embodiment of the invention a fabrication schematic of 5c with a single layered collagen-based sponge.

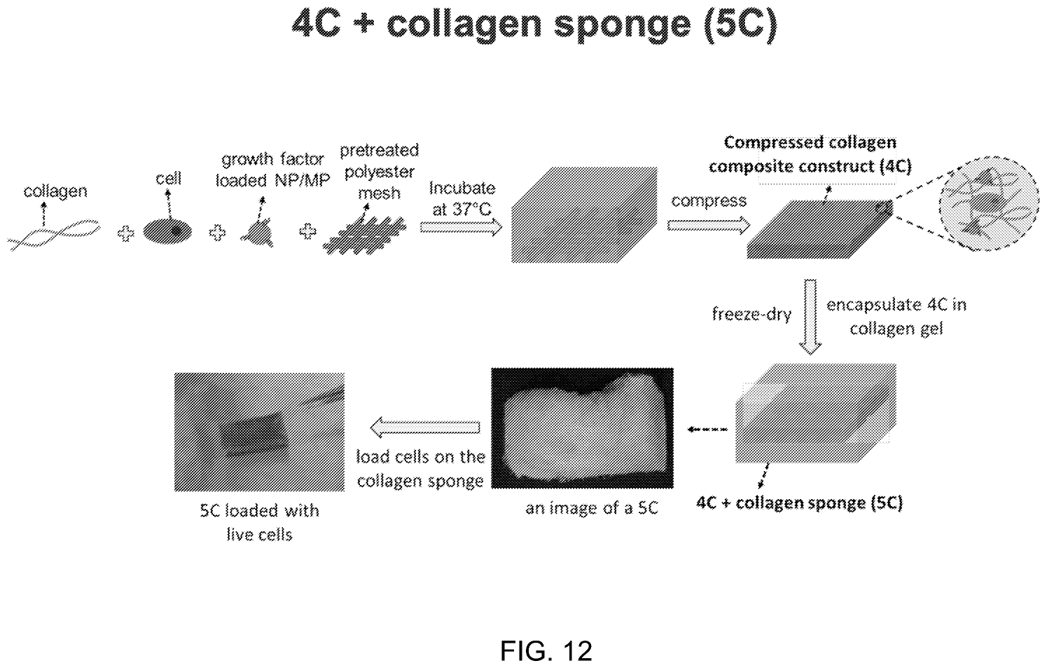

FIG. 13 shows according to an exemplary embodiment of the invention a fabrication schematic of 5c of variety, including not limited to a single or multiple layered collagen-based sponge with repeated steps.

FIG. 14 shows according to an exemplary embodiment of the invention thickness variation influenced by compression of the collagen hydrogel.

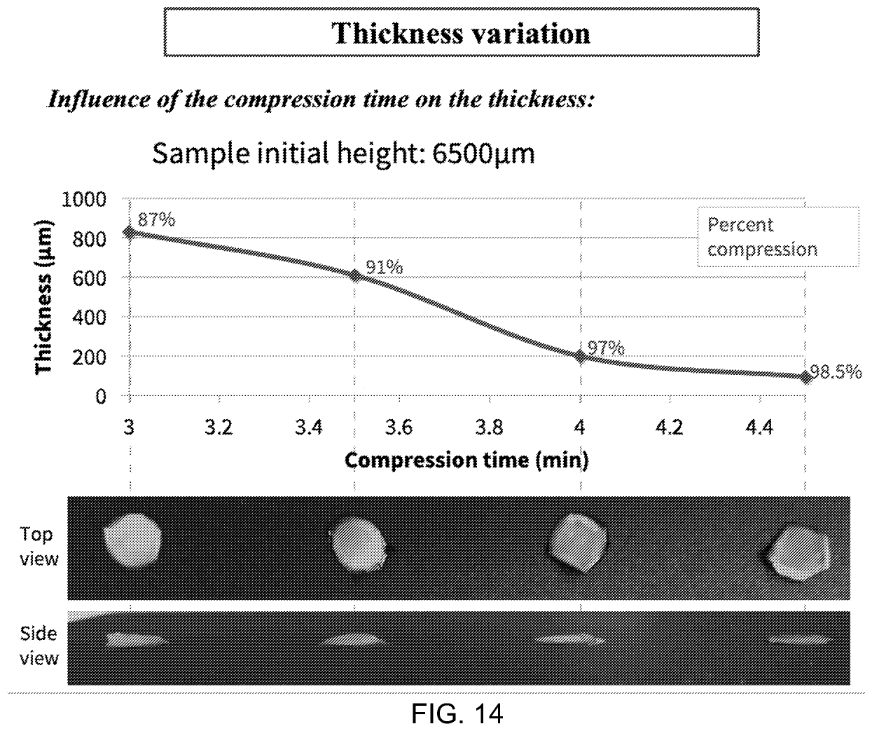

### DETAILED DESCRIPTION

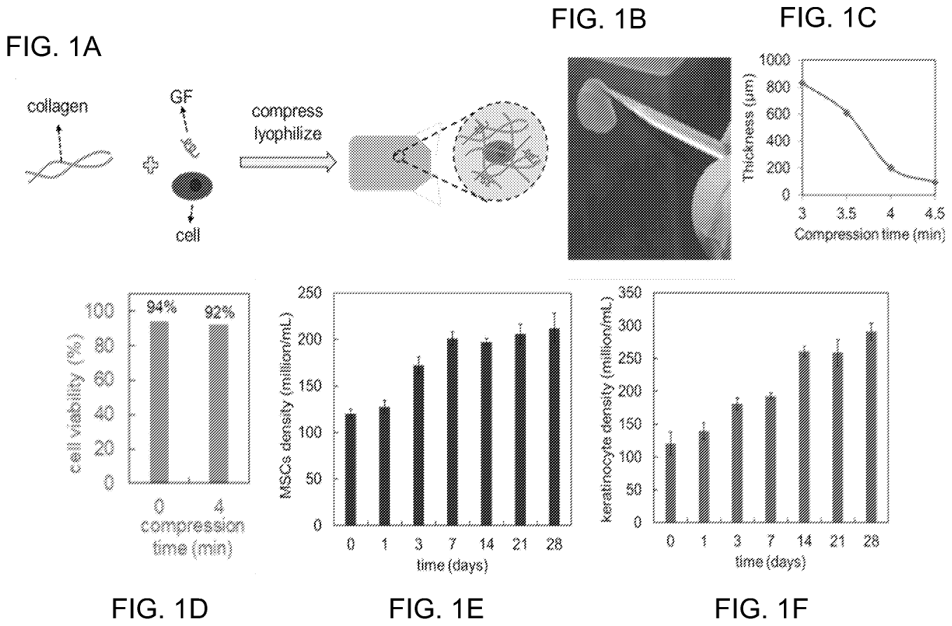

A solution of collagen or collagen fragment is prepared into which may be added harvested cells, bone marrow aspirate concentrates, growth factors, or growth factor loaded nanoparticles/microparticles (NPs/MPs) (FIGS. 1A-F). NPs/MPs include all inorganic and organic particles that are stable in the collagen solution in the 4° C. to 37° C. temperature range and 3-7.4 pH range. After adjusting the pH of the collagen solution to 7.4, it is cast in a mold and placed in an incubator at 37° C. and allowed to gel. The solidified gel is demolded and compressed gently between two layers of absorbent paper by applying a 2 mN/mm2 (0.2-200 mN/mm2 range) load for 1-6 minutes, or compressed by a device. The thickness of the collagen microsheet can be tuned with changing the compression time. The viability of cells in the compressed collagen was over 92%. When human mesenchymal stem cells (MSCs) and human keratinocytes were encapsulated in compressed collagen microsheet, the cell number increased by 1.8 and 2.4 folds, respectively (FIGS. 1A-F).

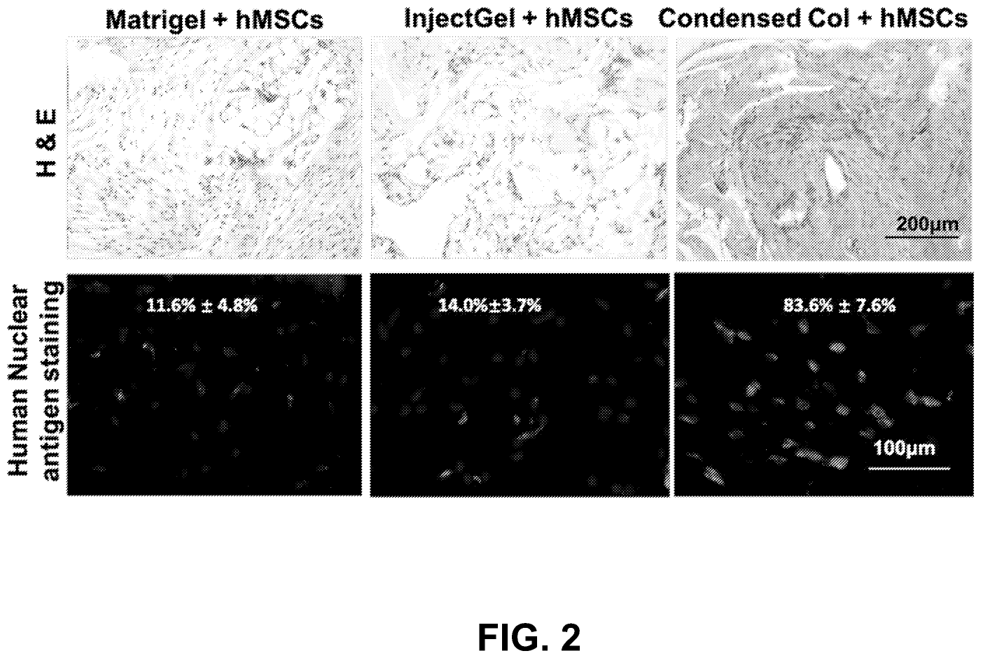

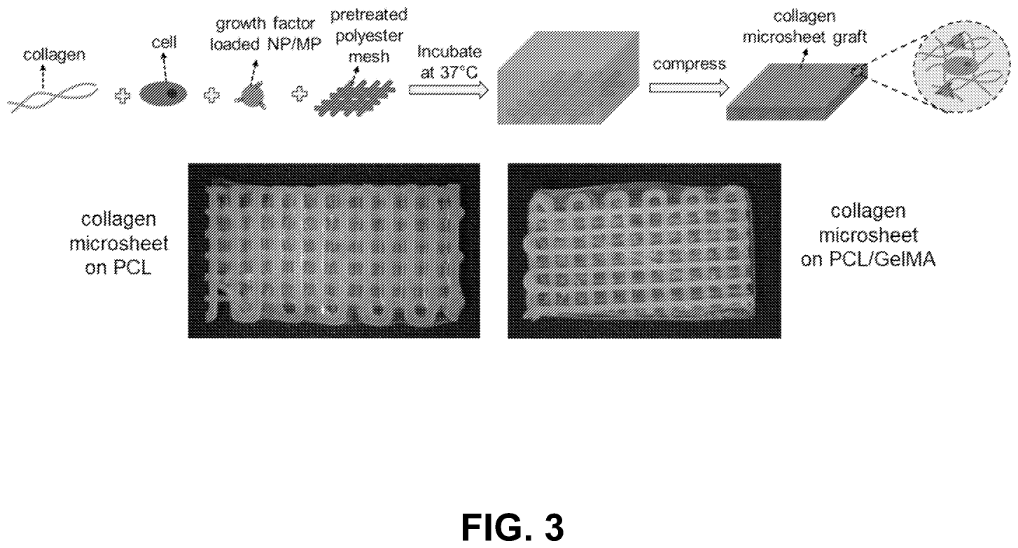

When compressed collagen was used for subcutaneous delivery of human MSCs to NSG mice, around 84% of the cells in the implantation site were human cells (FIG. 2). As a comparison, only 11% or 14% of the cells in the implantation site were human cells when Matrigel hydrogel or an injectable collagen-based hydrogel was used for cell delivery. The compressed collagen microsheet can be supported by a polymer mesh (FIG. 3). Addition of a polymer mesh mechanically supports the compressed collagen microsheet and facilitates manipulations such as bending, folding, rolling, stretching, and suturing. A range of polymers including polyesters (e.g. PCL, PLA, PLGA), polyurethanes, natural polymers (e.g. gelatin or methacrylated gelatin) and a range of manufacturing techniques including 3D printing and casting could be used to make the polymer mesh. For instance, a PCL or PCL/Gelatin Methacrylate mesh, was 3D printed and incorporated into collagen microsheet to make compressed collagen composite constructs (FIG. 3).

To incorporate a polyester mesh, the surface of the polyester is treated with NaOH (5 N) solution for 3 hours, followed by a treatment with ethyl-3-(3-dimethylaminopropyl)carbodiimide (EDC) and N-hydroxysuccinimide (NHS) solution (5 mg/mL) in MES buffer for 1 hour. he mesh is then washed with DI water and incubated in a GelMA or gelatin solution in MES buffer (2% wt/v) for 2 hours at room temperature. The GelMA- or gelatin coated mesh is then washed in DI water, dipped in EDC/NHS solution in MES buffer for 10 minutes at room temperature, washed again in DI water, and dried in a vacuum chamber. Half of a mold (could be Teflon, silicon, or other non-reactive polymers) is filled with collagen solution (pH 7.4) loaded with cells, growth factors or MPs/NPs. Then the mesh is placed in the mold, and the remaining space in the mold is filled with collagen solution (pH 7.4) loaded with cells, growth factors or MPs/NPs. Then, the mold is placed in an incubator at 37° C. and allowed to gel for 45 minutes. The solidified gel/mesh is demolded and compressed gently between two layers of absorbent paper by applying a 2 mN/mm2 load for 1-6 minutes, or compressed by a device.

The treatment of the mesh can be summarized as:
1. Making the mesh surface hydrophilic.
2. Adding interactive molecules.
3. Attach gelatin (which attach to the added interactive groups) to improve interaction with collagen.

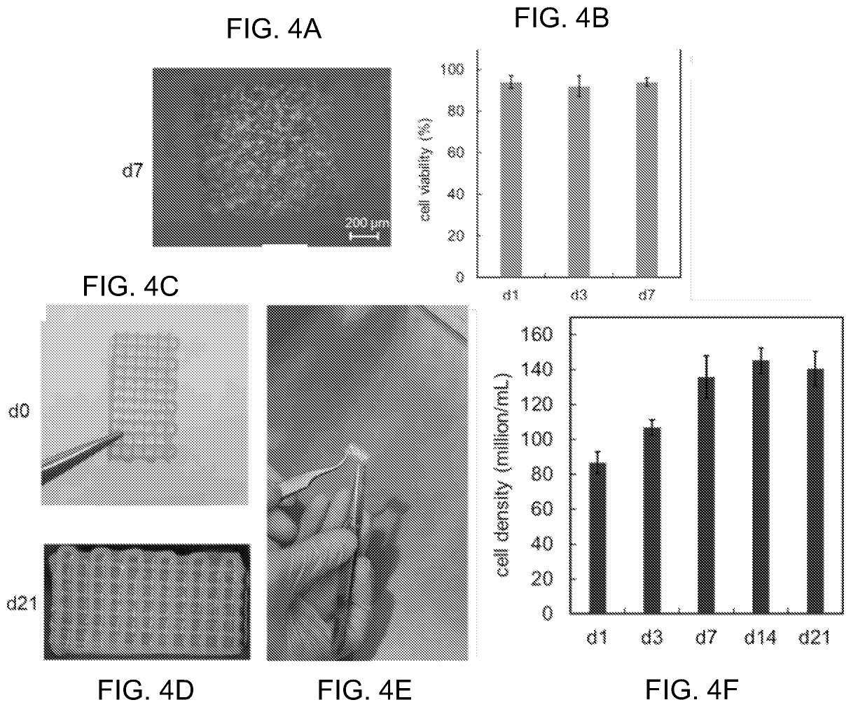

The method is compatible with cells, showing over 90% cell viability in 7 days after 4C construction (FIGS. 4A-B). The cell loading did not significantly affect the flexibility, physical characteristics, and manipulation of 4C constructs (FIGS. 4C-E). Also, the number of human MSCs encapsulated in 4C increased by 60% over 21 days (FIG. 4F).

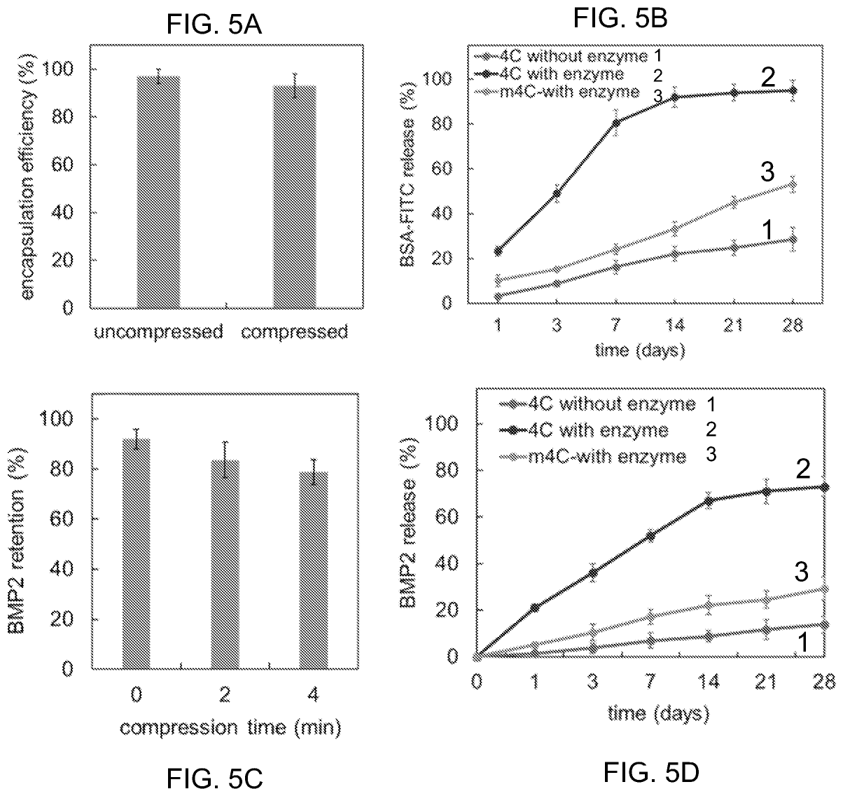

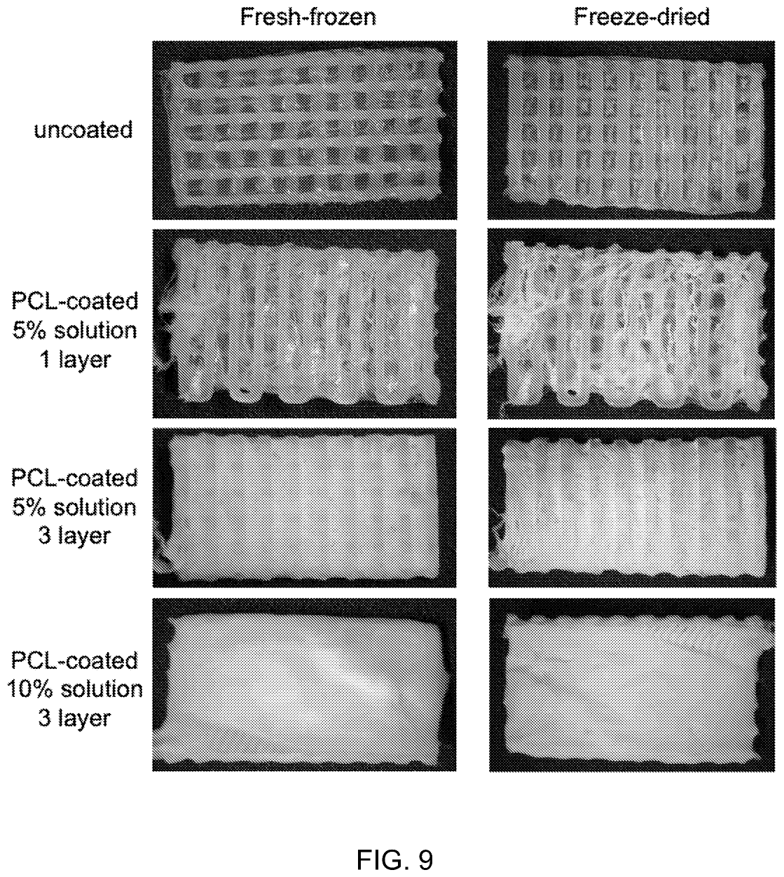

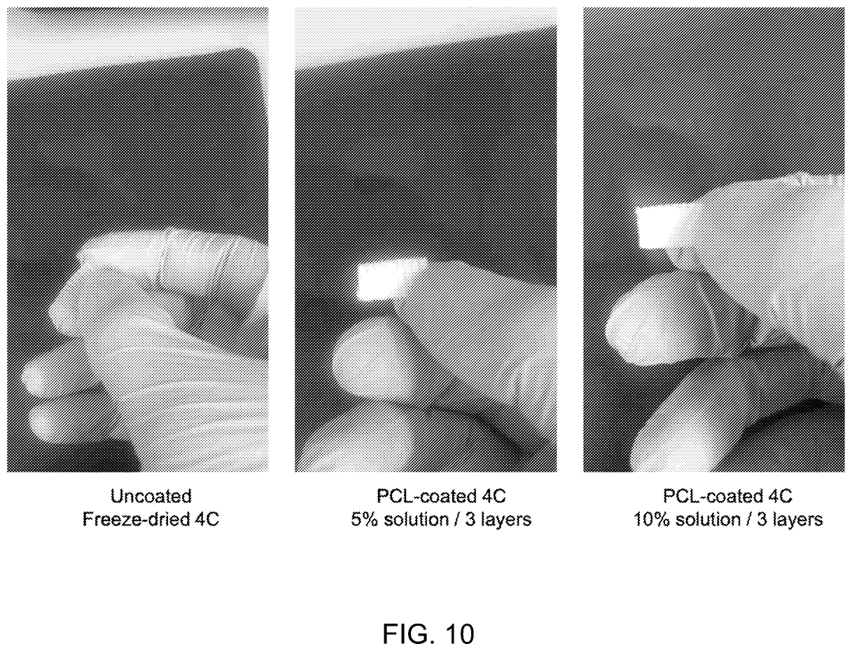

4C (Compressed Collagen Composite Construct) could also be used to deliver proteins. The amount of encapsulated BSA protein loss during the compression of 4C constructs was not significant (FIG. 5A). Also, the amount of retained BMP2 protein in 4C constructs after 4 minutes of compression was 79% (FIG. 5C). The encapsulated BSA protein in 4C had 28% release in PBS in the absence of collagenase enzyme over 28 days, while 92% of the BSA protein was released from 4C in 14 days in the presence of enzyme (FIG. 5B). To slow down the release, 4C constructs could be coated with a resorbable polyester (e.g. PCL, PLA, or PLGA) or other resorbable polymers (e.g. polyurethanes). For instance, protein-laden 4C was freezed at −80° C. and dipped in a solution of PCL in acetone for 30 seconds to deposit a layer of PCL on 4C and make modified 4C (m4C). Then the PCL-coated 4C was air-dried at 0-4° C. The concentration of PCL solution and the number of deposited PCL layers could be changed to tune the physical characteristics of m4C constructs and release kinetics of proteins (FIGS. 9-10). For instance, the amount of released BSA and BMP2 from m4C after 4 weeks in the presence of enzyme was 53% and 38%, respectively (FIG. 5B, 5D). Protein-laden devices may undergo freeze-drying and terminal sterilization to facilitate storage, transportation, and clinical translation Therefore, the effect of freeze-drying and E-beam sterilization on physical characteristics of 4C constructs was evaluated. The freeze-drying the E-beam sterilization did not dramatically impact the physical characteristics of 4C.

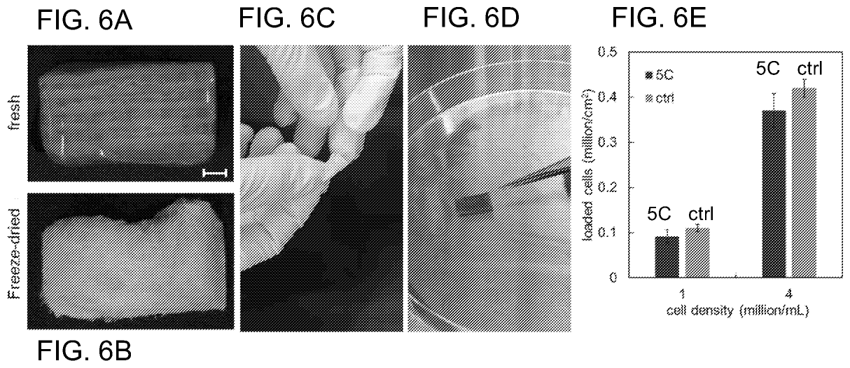

4C could be embedded in a spongy freeze-dried collagen to make 5C constructs (4C+collagen sponge, FIGS. 6A-B). 5C constructs are flexible and manipulatable, similar to 4C constructs (FIG. 6C). To make 5C, first Half of a mold is filled with collagen solution (pH 7.4). Then, a premade 4C is inserted in the mold. The mold is then filled with collagen solution (pH 7.4) and incubated in an incubator at 37° C. for 45 minutes. The 5C is then frozen at −80° C. and freeze-dried. Cells suspended in a medium can be directly loaded on 5C constructs. For instance, 200 L of hMSC cell suspension was loaded on 1 cm2 of 5C (FIG. 6D). There was not a significant cell loss after cell loading on 5C constructs, regardless of cell density in the suspension. (FIG. 6E).

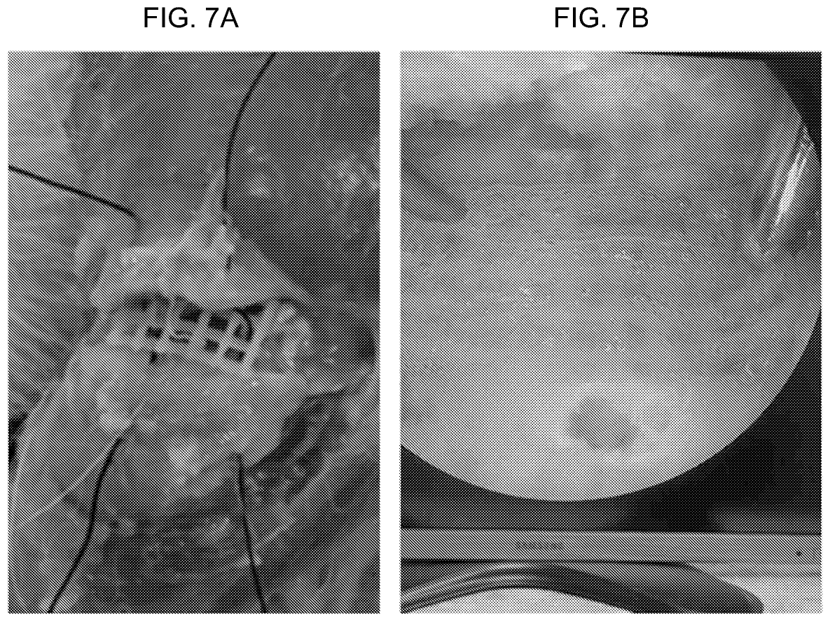

4C or 5C could find applications as drug delivery platforms and engineered tissue grafts for regenerative medicine for both soft and hard tissues. For drug/growth factor delivery, 4C or 5C is a practical delivery vehicle for drugs, growth factors, and NPs/MPs that can release their payload gradually through a combination of diffusion-mediated release and degradation-mediated release. For regenerative medicine applications, live cells could be delivered using 4C or 5C either through encapsulation in 4C or direct loading on a spongy collagen in 5C. Tissue engineering/regenerative medicine applications include rotator cuff repair, vascularized tissue flaps, cartilage repair, bone grafts, vascular grafts and spina bifida closure graft. The application of 4C or 5C constructs for rotator cuff repair was validated by grafting a 4C polymer mesh on a cadaveric rabbit rotator cuff defect model (FIG. 7A) and arthroscopically inserting a 5C in a human cadaveric shoulder (FIG. 7B).

Collagen is a naturally derived biocompatible biomaterial for therapeutic delivery. But under typical conditions the mechanical properties of collagen-based materials (e.g. gel, foam) are too weak for implantation, surgical handling or suturing. The method for collagen compression along with a polymer mesh that serves as a mechanical support in 4C or 5C enables the production of collagen-based grafts with sufficient flexibility and mechanical strength to be manipulated, implanted, and sutured. The presence of a polymer mesh in 4C or 5C specifically increases the mechanical strength and anchoring capabilities for load-bearing functions such as rotator cuff repair. To facilitate storage, transportation, and medical translation, the 4C or 5C constructs could be freeze-dried and sterilized using a radiation-based sterilization method (e.g. E-beam). Also, a modular design enables using different modes of delivery (e.g., growth factors in compressed collagen and live cells in collagen sponge in 5C). Surgeons may use 5C constructs to deliver patients own cells or bone marrow aspirate with or without other preloaded therapeutics.

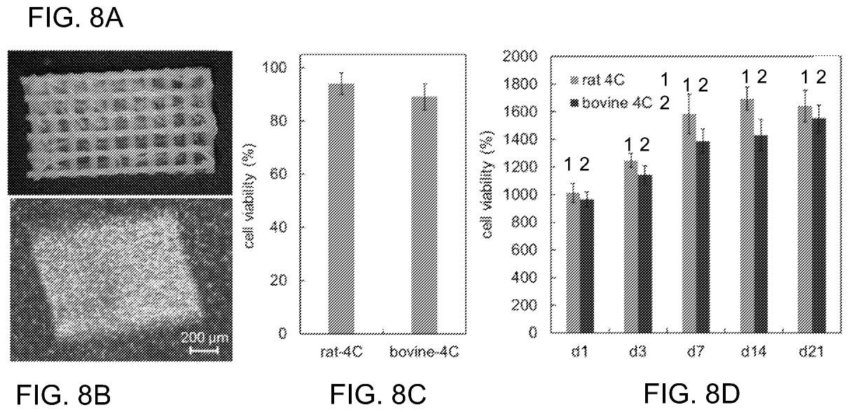

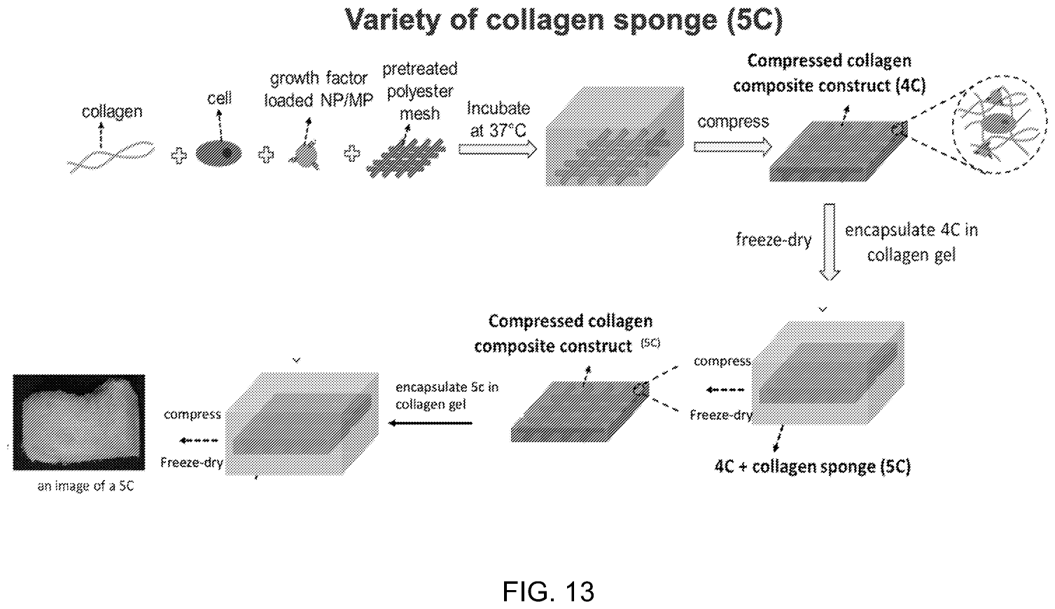

Compressed collagen composite constructs could be without (4C) or with (5C) a freeze-dried collagen sponge. A range of polymers including polyesters (e.g. PCL, PLA, PLGA), polyurethanes, natural polymers (e.g. gelatin or methacrylated gelatin) could be used to make the polymer mesh. Different types of collagen (e.g. type I collagen, type II collagen), and sources of collagen (e.g. rat, bovine, ovine, porcine, human) could be used to make 4C and 5C constructs. For instance, 4C or 5C constructs that have been reported herein were based on rat tail collagen. Bovine collagen was also used to make 4C constructs (FIG. 8A). The results showed that human MSCs were viable and proliferative in bovine-based 4C, similar to rat-based 4C (FIGS. 8B-D). Different types, sources, concentrations, or mixtures of cells, drugs, growth factors, NPs/MPs can be included in 4C or 5C depending on the desired application. To slow down the release of therapeutics and enhance the handleability of the composite construct is to repeat the collagen solution immersion+compression process+freeze-drying process (FIG. 13) to make the compressed collagen composite constructs (5C) with multi-layered freeze-dried collagen sponge. Another example to slow down the release of therapeutics, 4C constructs could be coated with a resorbable polyester (e.g. PCL, PLA, or PLGA) or other resorbable polymers (e.g. polyurethanes).

What is claimed is:

1. A method of making a therapeutic collagen-based delivery platform, comprising:
(a) having a mesh of struts of polymers;
(b) treating a surface of the mesh of struts of polymers to form a hydrophilic surface followed by coating with interactive molecular onto the surface of the mesh of struts for polymers to physically attach to the surface to enhance interaction with collagen, and wherein the treated mesh has a thickness of 0.2 to 3 mm;
(c) embedding the treated mesh in a collagen hydrogel forming a composite construct, wherein the collagen in the collagen hydrogel physically attaches to the polymers of the treated surface, wherein the collagen hydrogel has a thickness of 4 to 20 mm larger, and wherein the collagen hydrogel is loaded with therapeutics; and
(d) compressing the collagen hydrogel such that the composite construct has a thickness of 0.2 to 3 mm, plus not more than 300, 400, 600 or 900 micrometers.

2. The method as set forth in claim 1, further comprising freeze-drying the composite construct.

3. The method as set forth in claim 1, further comprising coating the composite construct.

4. The method as set forth in claim 1, further comprising embedding the composite construct in a sponge.

5. The method as set forth in claim 1, wherein the therapeutics are cells, drugs, proteins, peptides, small molecules, RNA, or particles loaded with drugs, proteins, small molecules, peptides, or RNA.

6. The method as set forth in claim 1, wherein the therapeutic collagen-based delivery platform produced by the method is used for regeneration or repair of an orthopedic injury or a soft tissue injury.

7. The method as set forth in claim 1, wherein the therapeutic collagen-based delivery platform produced by the method is used for regeneration or repair of a rotator cuff repair.

8. A therapeutic collagen-based delivery platform for regeneration or repair of an orthopedic injury or a soft tissue injury, comprising: a therapeutic collagen-based delivery platform, wherein the platform comprises a mesh of polymer struts having a hydrophilic surface and interactive surface molecules, wherein the mesh of polymer struts is embedded in a compressed collagen hydrogel, wherein the collagen in the collagen hydrogel is physically attached to polymers at the mesh surface through interactive molecules, wherein the thickness of the mesh of polymer struts is only increased by up to 300, 400, 600 or 900 micrometers as a result of the embedded and compressed collagen hydrogel.
